(19) United States
(12) Patent Application Publication
(10) Pub. No.: US 2002/0160503 A1
(43) Pub. Date: Oct. 31, 2002

(54) Method for the separation of adsorbents from microbiological growth media

(76) Inventors: Martin Jeffery Maresch, Wilmar, MN (US); Scott Reynolds Jeffrey, Raleigh, NC (US)

Correspondence Address: MYERS BIGEL SIBLEY & SAJOVEC, PO BOX 37428, RALEIGH, NC, 27627, US

(21) Appl. No.: 10/071,675

(22) Filed: Feb. 7, 2002

Related U.S. Application Data
(60) Provisional application No. 60/267,990, filed on Feb. 9, 2001.

(51) Int. Cl. C12N 1/20

(52) U.S. Cl. 435/252.3

(57) ABSTRACT

The present invention relates to a method for the separation of adsorbents from growth media, utilizing polymeric dispersants. These dispersants or flocculating agents have been found to efficiently separate beneficial adsorbents without significantly changing the viable microorganisms for further testing.

### RELATED APPLICATION

This application claims the benefit of United States Provisional Application No. 60/267,990, filed Feb. 9, 2001, the disclosure of which is incorporated herein by reference in its entirety.

### FIELD

The present invention relates to a method for separating an adsorbent material from a growth medium.

### BACKGROUND OF THE INVENTION

Adsorbents like charcoal or activated carbon are significant components in growth media. These adsorbents are known for their neutralizing and inhibitory properties and function as antimicrobial substances in media. Separation of these adsorbent components from the medium without removal of the microbial elements is a problem solved by embodiments of the present invention.

According to Thorpe et al., U.S. Pat. No. 5,162,229 and Thorpe and Weaver U.S. Pat. No. 5,314,855, it is standard practice to detect the presence of microorganisms in samples by culturing samples in a liquid growth medium. Medical test samples include body fluids such as blood, urine and cerebrospinal fluid (CSF). The detection of microorganisms in these samples can be impaired by the condition of the samples themselves. For example, medical samples may contain levels of antibiotics due to treatment regimens, and it is known that serum, plasma and lysed erythocytes and neutrophils contain natural microbial inhibitors. Industrial samples such as pharmaceuticals and foods may also contain antimicrobials or preservatives that inhibit the growth of microorganisms. Additionally, when culture media is prepared, autoclaving of the media at very high temperatures under pressure can result in the formation of by-products toxic to microorganisms. Removal or neutralization of these inhibitory or bactericidal substances is necessary to detect microbial contamination.

While adsorbents, such as charcoal or activated carbon serve an important role in the recovery of microorganisms, isolation of these microorganisms can be hindered by the presence of the adsorbents. Adsorbents may interfere with microscopic examination of the microorganisms and/or hamper the preparation of additional test suspensions, which require spectrophotometric techniques for standardization. Furthermore, the adsorbents may quench fluorescent compounds used in many microbiological tests.

Because of the relative particle sizes of the adsorbent to the microorganisms, physical methods of separation are inadequate. Centrifugation of the growth media can pelletize both the adsorbent and the microbes. Gravity settling of the adsorbents occurs very slowly due to small fines, which are suspended in the media. Filtration affords the removal of most of the adsorbent at the potential cost of removing the microorganisms as well.

Chemical methods may also lead to the imperfect separation of the adsorbent from the medium. Surfactants, which can enhance settling of adsorbents, are known to affect the cell membrane of certain microorganisms, elongating their shape and, subsequently, making further testing unreliable. Other types of molecules can bind to the surface of the cell, producing undesirable results in further analysis. Inorganic settling agents may not enhance the separation rates of the adsorbent from the medium. A nontoxic compound for the selective separation of the adsorbent had not been discovered for this use.

There are methods that focus on the separation or removal of carbon-based residues from an aqueous environment on a macro scale. Water treatment focuses on the settling of solids from large volumes of water. Various flocculants are added to the potential drinking water in ultra low concentrations. The flocculating agent cannot affect the quality of the water after treatment. The ecologically sound products produced by companies such as Nalco Chemical provide a safer alternative than those with potentially hazardous additives.

### SUMMARY OF THE INVENTION

The present invention relates to a method for the separation of adsorbents from liquid microbiological growth media using polymeric dispersants. These polymeric dispersants, or flocculating agents, have been found to efficiently separate beneficial adsorbents without significantly changing the level of viable microorganisms for further testing.

Presently, the advantage of adsorbent components in a culture medium is known to improve the recovery of various microorganisms. However, subsequent procedures are adversely affected by its presence. Known physical methods for their removal, such as centrifugation, lead to the reduction of viable microorganisms for further analysis. Chemical methods also have detrimental side affects, often affecting the morphology or viability of the organisms. Both current physical and chemical methods lead to adverse consequences to the quantity and/or quality of viable microorganisms in the growth media.

According to embodiments of methods of the present invention, an adsorbent is physically separated from a microbiological growth media that includes adsorbent and microorganisms by a polymeric dispersant, which preferentially interacts with the adsorbent over the microorganisms. The polymeric dispersant is preferably nontoxic.

According to the other embodiments of the methods of this invention, the microorganisms separated from the adsorbents become immediately available for further testing including direct smear methods, microbial identification, genotype analysis and susceptibility patterns. The methods may be used for assessing biological samples for medical tests, or industrial samples of food and the like.

Methods of the present invention may result in reduced laboratory processing cost, shorter time to obtain medically significant data, and/or more confidence in a positive result from a microbial analysis. Another benefit of the simplicity of the methods according to embodiments of the present invention is that they may easily lend themselves to implementation in conventional or existing laboratory procedures.

### DETAILED DESCRIPTION OF EMBODIMENTS OF THE PRESENT INVENTION

According to embodiments of the present invention, a method for the separation of adsorbents from growth media comprises the steps of taking a sample from the media, adding a polyacrylamide dispersant to said sample, and testing for the presence of any microorganisms present in said sample. The polyacrylamide dispersant preferably dissolves well in aqueous environments (e.g., blood) and then settles after flocculation. An exemplary dispersant is a copolymer of acrylamide and dimethylaminoethylacrylate methyl chloride quaternary salt (DMAEA.MCQ) or dimethylaminoethylmethacrylate methyl chloride quaternary salt (DMAEM.MCQ).

A preferred embodiment of the present invention is directed to a method as defined above wherein the copolymer is Ultimer® 1450/7750 (Nalco Chemical Company, Naperville, Ill.).

U.S. Pat. No. 6,025,426 to Hurlock et al., the disclosure of which is incorporated herein by reference in its entirety, describes such a copolymer of acrylamide and dimethylaminoethylacrylate methyl chloride quaternary salt (DMAEA.MCQ) or dimethylaminoethylmethacrylate methyl chloride quaternary salt (DMAEM.MCQ).

A quaternary salt can be any conventional quatemizing agent, as for example, methyl chloride. The polymers are commercially available from Nalco Chemical Co., Naperville, Ill. or may be prepared, as described in U.S. Pat. No. 6,025,426, as hydrophilic copolymers of acrylamide and dimethylaminoethylacrylate methyl chloride quaternary salt in a salt media containing a low molecular weight cationic dispersant polymer. Preferably, the chain transfer agents include sodium formate, isopropanol and 2-mercaptoethanol or similar compounds. Said polymer may be prepared by polymerization of acrylamide with DMAEA.MCQ or DMAEM.MCQ in a water/salt medium. In certain embodiments, the polyvalent anionic salt in the aqueous solution is suitably a sulfate, phosphate or mixture thereof. Preferred salts may be ammonium sulfate, sodium sulfate and the like. These salts may be each used as an aqueous solution thereof having a concentration of 15 percent or greater.

According to other embodiments of the present invention, a method for the separation of adsorbents from a growth medium comprises the steps of adding a polyacrylamide dispersant such as copolymer of acrylamide and dimethylaminoethylacrylate methyl chloride quaternary salt (DMAEA.MCQ) or dimethylaminoethylmethacrylate methyl chloride quaternary salt (DMAEM.MCQ) to the medium, and permitting the adsorbent to separate from the medium. After separation, the medium may be tested for microorganisms.

According to other embodiments of the present invention, a device for the enhanced recovery and detection of microorganisms and for continuously monitoring biological activity in a sample is provided. The device comprises a sealable, sterilizable, specimen container, having an internal chamber in which a sample comprising a microorganism may be cultured, the internal chamber enclosing a sample comprising a microorganism, a sterile culture medium, an adsorbent in an amount that is effective for neutralizing, binding, or inhibiting antimicrobial substances present in the sample and/or the medium, and a polymeric dispersant that interacts with the adsorbent. The container may have at least one transparent section therein and a sensor means located inside the container in the region of the transparent section. The sensor means may include a membrane and an indicator medium, the indicator medium being selected for its ability to exhibit a detectable change when exposed to products of an organism's metabolic activity or other changes in the environment, whereby changes in the appearance of the sensor means can be continuously monitored from the exterior of the container through the transparent section, thereby allowing for monitoring of biological activity without violating the integrity of the container after sealing. In certain embodiments, the present invention is also directed to instruments for the enhanced recovery and detection of microorganisms and for methods for detecting and continuously monitoring the growth of organisms using such instruments.

In certain embodiments, the polymeric dispersant interacts with the adsorbent in an aqueous medium (including blood) which causes the adsorbent to flocculate and settle. Thus, the polymeric dispersant is selected for its affinity for the adsorbent and for its reluctance to adversely affect the natural organism or microorganisms of interest. The polymeric dispersant may be selected based on its molecular weight and/or ability to coat the adsorbent to cause it to flocculate in a desired manner leaving the microorganisms substantially undisturbed in the growth media so that they are more easily discernible.

The following examples are given to further illustrate the features of the invention, but are not intended to limit the scope of the invention in any way.

### EXAMPLE 1

A standard group of microorganisms was tested (see Table 1) with a specific polyacrylamide copolymer dispersant named Ultimer® 1450/750 (Nalco Chemical Company, Naperville, Ill.). Microorganisms were grown under the appropriate temperature, atmosphere and media conditions, suspended in Trypticase Soy Broth at the appropriate concentration, and inoculated into BacT/Alert aerobic FAN™ bottles containing 10 ml of human blood. Bottles were loaded into the BacT/Alert™ instrument for monitoring and were removed from sampling upon being triggered as positive. Three subsets were generated from these bottles to prepare a comparison of the effect of Ultimer® 1450/7750. Three samples were aliquotted from each of the bottles as a means for a growth comparison study.

Sample A: An aliquot (0.5 ml) was removed from each bottle. Decimal dilutions were performed on the cell suspension. Samples were plated at 0.1 ml on the appropriated growth medium and incubated under the appropriate atmosphere and temperature conditions. Colonies were counted after overnight incubation for all organisms except for Micrococcus luteus 4698. These plates were placed in a sealed bag (to prevent drying) and incubated an additional 72 hours.

Sample B: Ultimer® 1450/7750 polyacrylamide (0.2 ml) was added to bottles after removing the aliquot for Sample A in the amount of 0.2 ml per bottle. The bottles were agitated to mix the agent and allowed to settle for approximately 5 minutes. After settling, bottles were sampled as described for Sample A plates with care taken to avoid re-suspending the settled charcoal. Decimal dilutions and plating were performed as described for the Sample A plates.

Sample C: This sampling was performed as described for the Sample B plates except that immediately prior to sampling, the bottles were vigorously agitated.

Table 1 shows the effects of the polymeric dispersant on the growth of a test panel of various microorganisms after a fixed time. Illustrated are (1) a duplicate of the untreated sample grown under the appropriate atmosphere, temperature and media conditions, (2) a duplicate of the settled adsorbent sample treated with polymeric emulsion which is grown under the appropriate atmosphere, temperature and media conditions, and (3) a duplicate of the resuspended adsorbent sample treated with polymeric emulsion grown under the appropriate atmosphere, temperature and media conditions. Populations of the microorganisms were established based on the colony counts from dilution factors of 10⁻⁷ to 10⁻³.

TABLE 1
Plate Counts for Pediatric Release Organisms

| Microorganism | | Sample A | | Sample B | | Sample C | |
|---|---|---|---|---|---|---|---|
| C. albicans | −5 | 179 | 163 | 79 | 97 | 113 | 119 |
| ATCC 14053 | Population | | 1.7E+07 | | 8.8E+06 | | 1.2E+07 |
| E. coli | −7 | 183 | 225 | 157 | 162 | 200 | 156 |
| ATCC 25922 | Population | | 2.0E+09 | | 1.6E+09 | | 1.8E+09 |
| H. influenzae | −7 | 30 | 26 | 30 | 35 | 23 | 25 |
| ATCC 10211 | Population | | 2.8E+08 | | 3.3E+08 | | 2.4E+08 |
| M. luteus | −7 | 24 | 26 | 22 | 16 | 168 | 248 |
| | −5 | | | | | | |
| ATCC 4698 | Population | | 2.5E+08 | | 1.9E+08 | | 2.1E+07 |
| N. meningitidis | −7 | 8 | 12 | 4 | 10 | 143 | 114 |
| | −5 | | | | | | |
| ATCC 13090 | Population | | 1.0E+08 | | 7.0E+07 | | 1.3E+07 |
| P. aeruginosa | −7 | 60 | 93 | 81 | 67 | 62 | 67 |
| ATCC 27853 | Population | | 7.7E+08 | | 7.4E+08 | | 6.5E+08 |
| S. agalactiae | −7 | 58 | 88 | 77 | 67 | 48 | 62 |
| ATCC 13813 | Population | | 7.3E+08 | | 7.2E+08 | | 5.5E+08 |
| S. aureus | −7 | 53 | 48 | 4 | 11 | 13 | 33 |
| ATCC 25923 | Population | | 5.1E+08 | | 7.5E+07 | | 2.3E+08 |
| S. epidermidis | −7 | 117 | 134 | 8 | 25 | 79 | 57 |
| ATCC 12228 | Population | | 1.3E+09 | | 1.7E+08 | | 6.9E+08 |
| S. pneumoniae | −7 | 12 | 6 | 1 | 0 | 1 | 0 |
| | −3 | | | | | | |
| ATCC 6305 | Population | | 9.0E+07 | | <1.0E+3 | | <1.0E+3 |
| X. maltophilia | −7 | 8 | 14 | 8 | 10 | 6 | 5 |
| ATCC 13637 | Population | | 1.1E+08 | | 9.0E+07 | | 5.5E+07 |
| S. pyogenes | −7 | 32 | 20 | 22 | 24 | 19 | 20 |
| ATCC 19615 | Population | | 2.6E+08 | | 2.4E+08 | | 2.0E+08 |

In this experiment, bottles were sampled between approximately 47.5 and 54.5 hours after loading into the BacT/Alert™ instrument. This represents a range of approximately 6 to 41 hours after being called positive. For S. pneumoniae 6305, sampling was performed approximately 40 hours after being called positive.

Only S. pneumoniae 6305 was adversely affected by the addition of Ultimer® 1450/7750. For the bacterial species tested except S. pneumoniae 6305, populations in excess of 10⁷ CFU/ml remained after the addition of Ultimer® 1450/7750 for Candida albicans 14053, a population of approximately 10⁷ CFU/ml remained after the addition of Ultimer® 1450/7750. Differences in the calculated population can be attributed to sampling errors. For S. pneumoniae 6305, the difference in population was dramatic with greater than a 4 log reduction after the addition of Ultimer®t 1450/7750. S. pneumoniae is known to undergo autolysis as the culture ages. The second experiment was under taken to determine the effect of age and handling on the recovery of the organism.

### EXAMPLE 2

In another experiment, S. pneumoniae 6305 was tested at two different times. The microorganism was grown under the same conditions as Example 1 and inoculated into 2 duplicate sets of aerobic FAN™ Aerobic bottles containing 10 ml of human blood. Two bottles were removed and sampled promptly after being called positive by the BacT/Alert™ instrument. The other two were removed and sampled to correspond to the time frame in the initial experiment. In each case, one bottle was treated as described for the initial experiment. The second bottle was treated the same except that no Ultimer® 1450/7750 was added to the bottle.

This table shows the effect of time on the re-growth of this microorganism. Under the same conditions in Table 1, S. pneumoniae—ATCC 6305 was used in 2 duplicate sets; one immediately upon triggering a positive response by the BacT/Alert™ instrument, and another after an extended time. Colony counts from dilution factors of 10⁻⁷ to 10⁻¹ are used to calculate the colony population in the samples.

TABLE 2
Plate Counts for S. pneumoniae - ATCC 6305

| Fresh | | | | | | Old | | | | | | | |
|---|---|---|---|---|---|---|---|---|---|---|---|---|---|
| | | Sample A | | Sample B | | Sample C | | Sample A | | Sample B | | Sample C | |
| Counts at | −7 | 221,234 | | | | | | | | | | | |
| | | 218,251 | | | | | | | | | | | |
| Dilution | −6 | | | | | | | | | | | | |
| Factor | | | | | | | | | | | | | |
| (10^{x}) | −5 | | | >1000 | >1000 | >1000 | >1000 | | | | | | |
| | −3 | | | | | | | | | | | | |
| | −2 | | | | | | | | | 1,0 | | 1,1 | |
| | −1 | | | | | | | 0,0 | 0,0 | 5,8 | 0,0 | 3,5 | 0,0 |
| Population | | 2.30E09 | 2.30E09 | >1E08 | >1E08 | >1E08 | >1E08 | <10 | <10 | 60 | <10 | 70 | <10 |

In this experiment, S. pneumoniae 6305 was originally sampled approximately 5 hours after being called positive. The second set of bottles was sampled approximately 34 hours after being called positive.

In this experiment, the “fresh” cultures showed no appreciable decrease in population. For the “old” cultures, the organisms had apparently undergone autolysis prior to sampling.

### EXAMPLE 3

A method for the separation of adsorbents from liquid microbiological growth media includes the following steps:

a) Aseptically remove 3 mL of test sample from a charcoal containing blood culture bottle.

b) Place the aliquot in a sterile container.

c) Insert a sterile 10 μL loop into the dispersant that comprises a copolymer of acrylamide and dimethylaminoethylacrylate methyl chloride quaternary salt (DMAEA.MCQ) or dimethylaminoethylmethacrylate methyl chloride quaternary salt (DMABM.MCQ). Remove the loop slowly to withdraw approximately 10 μL of dispersant, for a dilution ratio of 1 part dispersant to 300 parts test sample (1:300). Dispersant dilutions from 1:150 to 1:500 may be used in this procedure.

d) Insert the loop directly into the test sample and mix the sample with the loop for 5-10 seconds.

e) Discard the loop, replace the cap and invert the tube several times.

f) Place the tube upright in a rack and allow charcoal to settle for approximately 5 minutes.

g) Remove samples for Gram staining or further investigation from the uppermost level in the tube.

### EXAMPLE 4

An alternative method for the separation of adsorbents from liquid microbiological growth media includes the following steps:

a) Prepare a 1:400 dilution of the dispersant, which comprises a copolymer of acrylamide and dimethylaminoethylacrylate methyl chloride quaternary salt (DMAEA.MCQ) or dimethylaminoethylmethacrylate methyl chloride quaternary salt (DMAEM.MCQ), by adding 100 μL of dispersant reagent to 40 mL of sterile distilled water. Mix by gentle agitation.

b) Add 3 mL of the diluted dispersant to a clear sterile screw-capped tube (e.g., 100×12.5 mm).

c) Add 3 mL of test sample from the charcoal containing culture bottle. Dispense sample down the side of the tube.

d) Replace cap and invert the tube gently several times until charcoal clumping is observed.

e) Allow the tube to stand upright undisturbed for a minimum of 2 minutes and a maximum of 5 minutes.

f) OPTIONAL: Tubes may be centrifuged briefly (1 minute at 800-1,000 rpm) to further pellet the charcoal.

g) Collect the charcoal depleted supernatant for Gram staining or further investigation.

In the specification and examples, there have been disclosed typical preferred embodiments of the invention and, although specific terms are employed, they are used in a generic and descriptive sense only and not for purposes of limitation of the scope of the invention set forth in the following claims.

That which is claimed is:

1. A method for testing for microorganisms in a growth medium comprising a microorganism and an adsorbent, said method comprising the steps of:
providing a sample of the growth medium that comprises a microorganism and an adsorbent;
adding a polyacrylamide dispersant to the sample;
permitting the adsorbent to separate from the medium; and
testing the growth medium from which the adsorbent has been separated for the presence of the microorganism.

2. A method according to claim 1 wherein the polyacrylamide dispersant is a copolymer of acrylamide and dimethylaminoethylacrylate methyl chloride quaternary salt (DMAEA.MCQ) or a copolymer of acrylamide and dimethylaminoethylmethacrylate methyl chloride quaternary salt (DMAEM.MCQ).

3. A method according to claim 1 wherein the adsorbent comprises charcoal.

4. A method according to claim 1 wherein the adsorbent is charcoal.

5. A method according to claim 1 wherein the growth medium is an aqueous growth medium.

6. A method according to claim 1 wherein the growth medium comprises blood.

7. A method according to claim 1 wherein the microorganism is selected from the group consisting of C. albicans, E. coli, H. influenzae, M. luteus, N. meningitidis, P. aeruginosa, S. agalactiae, S. aureus, S. epidermidis, S. pneumoniae, X. maltophlia, and S. pyogenes.

8. A method for the separation of an adsorbent from a growth medium comprising the adsorbent, said method comprising the steps of:
adding a polyacrylamide dispersant to the growth medium; and
permitting the adsorbent to separate from the medium.

9. A method according to claim 8 wherein the polyacrylamide dispersant is a copolymer of acrylamide and dimethylaminoethylacrylate methyl chloride quaternary salt (DMAEA.MCQ) or a copolymer of acrylamide and dimethylaminoethylmethacrylate methyl chloride quaternary salt (DMAEM.MCQ).

10. A method according to claim 8 wherein the adsorbent comprises charcoal.

11. A method according to claim 10 wherein the adsorbent is charcoal.

12. A method according to claim 8 wherein the growth medium is an aqueous growth medium.

13. A method according to claim 8 wherein the growth medium comprises blood.

14. A method according to claim 8 wherein the growth medium comprises a microorganism.

15. A method according to claim 1 wherein the microorganism is selected from the group consisting of C. albicans, E. coli, H. influenzae, M. luteus, N. meningitidis, P. aeruginosa, S. agalactiae, S. aureus, S. epidermidis, S. pneumoniae, X. maltophlia, and S. pyogenes.

16. A device for the enhanced recovery and detection of microorganisms, said device comprising a sealable, sterilizable, specimen container, having an internal chamber in which a sample comprising a microorganism may be cultured, the internal chamber enclosing a sample comprising a microorganism, a sterile culture medium, an adsorbent in an amount that is effective for neutralizing, binding, or inhibiting antimicrobial substances present in said sample and said medium, and a polymeric dispersant that interacts with the adsorbent, the container having at least one transparent section therein.

17. A device according to claim 16 further comprising a sensor means located inside said container in the region of the transparent section such that an indication from the sensor means is visible through the transparent section.

18. A device according to claim 17 wherein said sensor means comprises a membrane and an indicator medium, the indicator medium being selected for its ability to exhibit a detectable change when exposed to products of an organism's metabolic activity.

19. A device according to claim 18 whereby changes in the appearance of the sensor means can be continuously monitored from the exterior of said container through said transparent section, thereby allowing monitoring of biological activity without violating the integrity of said container after sealing.

20. A device according to claim 16 wherein the polyacrylamide dispersant is a copolymer of acrylamide and dimethylaminoethylacrylate methyl chloride quaternary salt (DMAEA.MCQ) or a copolymer of acrylamide and dimethylaminoethylmethacrylate methyl chloride quaternary salt (DMAEM.MCQ).

21. A device according to claim 16 wherein the adsorbent comprises charcoal.

22. A device according to claim 16 wherein the adsorbent is charcoal.

23. A device according to claim 16 wherein the growth medium is an aqueous growth medium.

24. A device according to claim 16 wherein the growth medium comprises blood.

25. In a method for cultivating microbial organisms in a liquid growth medium to which an adsorbent has been added to adsorb antimicrobial materials, an improvement comprising adding a polymeric dispersant to the growth medium to precipitate the adsorbent.

26. A method according to claim 25 wherein the polyacrylamide dispersant is a copolymer of acrylamide and dimethylaminoethylacrylate methyl chloride quaternary salt (DMAEA.MCQ) or a copolymer of acrylamide and dimethylaminoethylmethacrylate methyl chloride quaternary salt (DMAEM.MCQ).

27. A method according to claim 25 wherein the adsorbent comprises charcoal.

28. A method according to claim 25 wherein the adsorbent is charcoal.

29. A method according to claim 25 wherein the growth medium is an aqueous growth medium.

30. A method according to claim 25 wherein the growth medium comprises blood.

31. A method according to claim 25 wherein the microorganism is selected from the group consisting of C. albicans, E. coli, H. influenzae, M. luteus, N. meningitidis, P. aeruginosa, S. agalactiae, S. aureus, S. epidermidis, S. pneumoniae, X. maltophlia, and S. pyogenes.
